(19) United States
(12) Patent Application Publication
(10) Pub. No.: US 2024/0358306 A1
(43) Pub. Date: Oct. 31, 2024

(54) WEARABLE ELECTROCARDIOGRAM DEVICE AND METHODS OF USE THEREOF

(71) Applicant: DRS.LINQ, Inc., Newtown, PA (US)

(72) Inventor: Rakesh Shah, Newtown, PA (US)

(21) Appl. No.: 18/650,008

(22) Filed: Apr. 29, 2024

Related U.S. Application Data
(60) Provisional application No. 63/499,123, filed on Apr. 28, 2023.

(51) Int. Cl. A61B 5/257; A61B 5/282

(52) U.S. Cl. CPC A61B 5/257; A61B 5/282

(57) ABSTRACT

The present disclosure relates to an electrode patch. The electrode patch includes a substrate and a plurality of electrodes mounted on the substrate. The electrodes are configured to collect electrocardiogram (ECG) measurements from a subject. The substrate includes a central portion that includes a concave side that is shaped and dimensioned such that upon alignment of the concave side under a breast of the subject, the plurality of electrodes are accurately positioned for collecting ECG measurements from the subject.

### RELATED APPLICATIONS AND CLAIMS OF PRIORITY

This application claims priority to U.S. Provisional Application No. 63/499,123 filed Apr. 28, 2023, the disclosure of which is incorporated herein in its entirety.

### FIELD OF THE DISCLOSURE

The present disclosure relates generally to the field of medical devices. More specifically, the present disclosure relates to an Electrocardiogram (“ECG”) system.

### BACKGROUND OF THE DISCLOSURE

Over 1.1 million heart attack subjects and 8-10 million chest discomfort subjects visit an emergency room every year. Visiting the emergency room can result in costly hospital stays. It is estimated that 90-95% of chest pain visits are medically unnecessary and result in losses of billions of dollars between subject cost and lost work. Additionally, there are approximately 17 million subjects that have major cardiac risk factors such as: diabetes, high blood pressure, high cholesterol, or smoking.

An electrocardiogram (ECG) is a test that can check for issues with a heart by recording electrical activity in the heart. The electrical activity can be recorded over a period of time by electrodes placed on a subject's body. The electrodes detect electrical changes on the skin that arise from depolarization of the heart during each heartbeat. The electrodes can be coupled to a device that processes the recorded electrical activity, and/or displays the activity as a waveform which can then be analyzed to help treat health problems. ECG's can also be used to analyze and monitor the effectiveness of medication or other implanted medical devices. An ECG is generally performed with the use of four peripheral electrodes placed on the limbs, and six electrodes placed on the precordium, i.e., the subject's chest. The peripheral electrodes, (limb leads), are designated left arm, right arm, left leg and right leg while the precordial electrodes are designated V1, V2, V3, V4, V5, and V6 to indicate the positions on the precordium on which the electrodes are placed. The limb leads are used to determine the electrical axis (to generate an output of 6 channels which are labelled aVR, aVL, aVF, I, II and III leads) which is usually measured only in the coronal plane, while the precordial electrodes are used to record the ECG in the transverse or horizontal plane. The electric activity is recorded and traditionally displayed as an electrocardiogram, exhibiting a characteristic waveform, illustrating the contractions of the heart atria and ventricles, their frequency and duration. Deviations from the normal pattern can be potentially pathological and therefore of clinical significance.

Typically, electrocardiograms (ECGs) are performed in a clinical setting by trained clinicians, such as registered nurses, doctors, nurses' assistants, or ECG technicians. To perform an ECG, the clinician may place electrodes in specific anatomical locations on the subject (i.e., on the precordium and the limbs). For a standard 12-lead electrocardiogram, there may be ten or more electrodes that must be placed. Once the electrodes are placed, the clinician may connect each electrode with its corresponding cable. As such, electrocardiograms (even when performed by a trained clinician) are often subject to human error due to misplaced electrodes and/or mismatched cables. The 12-lead placement model is also poorly suited to long-term ambulatory monitoring both from the perspective of comfort and from the perspective of reliability.

Moreover, it is often desirable to perform continuous, ambulatory ECG recording outside of a clinical setting. This could be the case for subjects where heart problems are suspected, or subjects recovering from an infarct. Subjects investigated for receiving a pacemaker are another group where continuous, ambulatory ECG recording is desired. Yet another group of subjects requiring an improved, yet more simple and easily performed ECG, are subjects in transit, e.g., in emergencies, during ambulance transport to a hospital, or during transfer within or between hospitals. Further, there is a growing interest for home monitoring of various health parameters, such as ECG. At home devices and systems for ECGs have become more and more prevalent in recent years with the increase in use of mobile and smart devices. At home devices can produce results similar to those achieved in a physician's office or emergency room for a fraction of the cost and reduce indirect costs such as lost workday. At home devices can also lead to early detection of cardiac issues thus improving treatment options and further reducing cost.

Such at home devices currently rely on a Holter monitor for performing mobile ECGs. This requires fixing multiple leads on to the subject's chest and carrying around the cumbersome device that only has a few hours power. A Holter monitor also needs to be fitted to the subject by a trained clinician, and due to the uncomfortable and non-ergonomic design, the subjects are reluctant to wear the device for the necessary long periods to identify any intermittent or transient pathologies. Specifically, Holter monitors can only be used for rhythm monitoring but do not provide enough data to assess for a heart attack. In addition, the data generated by the Holter monitor are not accessible in real time to provide a diagnosis.

The current disclosure describes devices and methods directed towards mobile ECG devices.

### SUMMARY OF THE DISCLOSURE

The present disclosure relates to an electrode patch. In a first scenario, the electrode patch includes a substrate including a base surface; and a plurality of electrodes mounted with respect to the base surface. The electrodes are configured to collect electrocardiogram (ECG) measurements from a subject when the base surface is placed against a precordium of the subject. The substrate includes a central portion that includes a concave side that is shaped and dimensioned such that when the base surface is placed against a precordium of the subject.

Optionally, the concave side has a radius of curvature that is similar to a radius of curvature of webspace between a thumb and an index finger of a hand of the subject when the hand is open. Optionally, the concave side has a radius of curvature that is between about 1 to 4 inches.

Optionally, the electrode patch includes a first peripheral portion and a second peripheral portion that flank the central portion. Optionally, the first peripheral portion and the second peripheral portion flank the central portion to form an approximate L-shape. Optionally, the approximate L-shape mimics a shape formed by an index finger and a thumb when a hand of the subject is open.

Optionally, the first peripheral portion has a length that is similar to an average size of a thumb of a human subject, and the second peripheral portion has a length similar to an average size of a finger of the human subject.

Optionally, the concave edge has an arc angle is configured to at least partially concentrically align with an underside of a breast (i.e., a left breast) of the subject. Optionally, arc angle is approximately the same angle as an arc angle of webspace between a thumb and an index finger of the subject when a hand of the subject is open.

Optionally, the electrode patch includes at least four electrodes for collection of ECG measurements from one or more precordial locations on the subject's chest.

Optionally, the electrode patch includes four electrodes for collection of ECG measurements from the following precordial locations: V2, V3, V4, and V5.

Optionally, the electrode patch is expandable to include at least one more electrode for collection of ECG measurements from one or more of the following precordial locations: V1 and V6.

Optionally, the electrode patch includes a support configured to allow the subject to hold the electrode patch in place. Optionally, the support includes a loop configured and dimensioned to receive a thumb of the subject, the loop included in a first peripheral portion flanking the central portion. Optionally, the support includes a second loop configured and dimensioned to receive a different finger of the subject, the second loop included in a second peripheral portion flanking the central portion.

Optionally, the support includes a finger cup configured and dimensioned to receive a thumb of the subject and that is included in a first peripheral portion flanking the central portion. Additionally, the support can include a second finger cup configured and dimensioned to receive one or more different fingers of the subject and that is included in a second peripheral portion flanking the central portion.

Optionally, the electrode patch further includes a comfort lip extending from the concave side and that provides padding to the breast of the subject upon placement of the concave edge under the breast of the subject. Optionally, the comfort lip is further configured to extend at least partially over a webspace between a thumb and an index finger of a hand of the subject for holding or positioning the electrode patch under the breast of the subject. Optionally, the concave edge forms an inner curved surface. In various embodiments, a size or a location of the comfort lip is configured to allow the subject to correctly position webspace between a thumb and an index finger of a hand of the subject when the hand is open while holding the electrode patch in place.

Optionally, the electrode patch includes a structural element configured to allow the subject to correctly position webspace between a thumb and an index finger of a hand of the subject when the hand is open while holding the electrode patch in place.

In some embodiments, the substrate further includes one or more hinges for folding at least two portions of the substrate on top of each other. Optionally, the support is made from a rigid reusable material.

In another scenario, the present disclosure relates to an electrode patch that includes a substrate configured for placement against at least a portion of a precordium of a subject, a support connected to the substrate and configured and configured to naturally assume a curvature in an unloaded state; and a plurality of electrodes mounted on the substrate and configured for collecting electrocardiogram (ECG) measurements from the subject.

Optionally, substrate is configured to allow the subject to hold the substrate partially around the breast of the subject.

In one or more embodiments, the support includes a loop configured and dimensioned to receive one or more fingers of the hand of the subject. Optionally, the substrate has an approximate L shape that is sized similarly to a natural L shape of an index finger and a thumb of the hand of the subject when the hand of the subject is open, the loop being configured to extend around the thumb of the subject. Optionally, the support includes a second loop configured and dimensioned to receive the index finger of the subject.

In some embodiments, the support is a comfort lip extending from a surface of the substrate and covers at least the portion of the hand of the subject to provide padding to the breast of the subject. Optionally, the comfort lip is continuous along an edge of the substrate. Optionally, the comfort lip includes a hand contacting surface configured to extend over the hand of the subject and a breast contacting surface configured to extend under the breast of the subject and provide a comfortable place for the breast to rest. Optionally, the comfort lip has an approximate U-shape, wherein a concave portion of the approximate U-shape is the hand contacting portion, and a convex portion of the approximate U-shape is the breast contacting portion.

Optionally, the substrate includes a central portion including a concave edge that is shaped and dimensioned such that upon alignment of the concave edge adjacent the breast of the subject, the plurality of electrodes are accurately positioned for collecting ECG measurements.

In another scenario, the present disclosure relates to an electrocardiogram (ECG) lead system. The ECG lead system includes up to four precordial electrodes and a number of the precordial electrodes being sufficient to generate a complete and accurate ECG of a subject. The ECG lead system also includes one or more limb electrodes electrically coupled to the precordial electrodes, and a plurality of electrical pathways connecting the precordial electrodes and the one or more limb electrodes. The precordial electrodes and the one or more limb electrodes are configured to electrically connect to a data collection device which collects data from each of the electrodes. Optionally, the data being sufficient to generate a complete and accurate ECG.

Optionally, the ECG lead system comprises the following four precordial electrodes are: V2, V3, V4, and V5 electrodes.

Optionally, the four limb electrodes are: right arm, left arm, right leg, and left leg electrodes.

Optionally, the four precordial electrodes are mounted on a substrate of an electrode patch.

Optionally, the substrate includes a central portion including a concave side that is shaped and dimensioned such that upon alignment of the concave side at least partially around a breast of a subject, the four precordial electrodes are accurately positioned for collecting ECG measurements.

Optionally, the electrode patch includes a structural element that includes a support mounted to the substrate configured to allow a subject to hold the electrode patch in place.

In another scenario, the present disclosure relates to a diagnostic system that includes the ECG lead system described above, the data collection device electronically coupled to the ECG lead system, a user device in electronic communication with the data collection device, and a network in electronic communication with the user device and a remote server.

Optionally, the data collection device receives data from the ECG lead system and communicates the data to the remote server, and the remote server communicates an ECG to the user device.

In yet another scenario, the current disclosure relates to an electrode patch that includes an L-shaped substrate, and a plurality of electrodes mounted on a base surface of the substrate and configured to collect electrocardiogram (ECG) measurements from a subject. The substrate also includes at least one structural element oriented facing away from the base surface.

In some embodiments, the at least one structural element is configured to allow the subject to hold the electrode patch against a pericardium of the subject. Optionally, the at least one structural element includes a loop configured and dimensioned to receive a thumb of the subject and that extends from a first arm of the L-shaped substrate. Additionally, the at least one structural element can include a second loop configured and dimensioned to receive a different finger of the subject and that extends from a second arm of the L-shaped substrate.

In some embodiments, the at least one structural element includes one or more flaps extending from an inside edge of the L-shaped substrate. The one or more flaps are configured to extend at least partially over a webspace between a thumb and an index finger of a hand of the subject for holding or positioning the electrode patch under the breast of the subject. Optionally, the electrode patch may include one flap that extends from a central concave portion of the inside edge of the L-shaped substrate. Optionally, the electrode patch may include two flaps that each extend from a first arm and a second arm respectively of the L-shaped substrate.

In certain embodiments, the at least one structural element is a comfort lip configured to provide padding to a breast of the subject upon placement of the L-shaped substrate around a breast of the subject. Optionally, the comfort lip extends from an inside edge of the L-shaped substrate and is further configured to extend at least partially over a webspace between a thumb and an index finger of a hand of the subject for holding or positioning the electrode patch under the breast of the subject.

In certain other embodiments, the at least one structural element is configured to allow the subject to correctly position webspace between a thumb and an index finger of a hand of the subject when the hand is open while holding the electrode patch against a precordium of the subject. Optionally, the at least one structural element includes a finger cup configured and dimensioned to receive a thumb of the subject, the finger cup included in a first arm of the L-shaped substrate. Additionally, the at least one structural element includes a second finger cup configured and dimensioned to receive one or more different fingers of the subject, the second finger cup included in a second arm of the L-shaped substrate.

In another scenario, the present disclosure relates to an electrode patch that includes a substrate having a base surface, a plurality of electrodes mounted with respect to the base surface and configured to collect electrocardiogram (ECG) measurements from a subject when the base surface is placed against a precordium of the subject, and at least one electrode mounted on a hand facing surface of the substrate. The hand facing surface is in contact with a hand of the subject when holding the base surface against the pericardium of the subject.

Optionally, at least one sensor can be mounted on the hand facing surface. The at least sensor may be, for example, a temperature sensor, an accelerometer, or a pulse oximeter.

Optionally, the substrate may be L-shaped.

In some scenarios, a diagnostic method for collecting electrocardiogram (ECG) data from a subject is disclosed. The method includes providing an ECG lead system including up to four precordial electrodes mounted on a substrate where a number of the precordial electrodes is selected to generate a complete and accurate ECG of a subject. The method further includes positioning a concave side the substrate around an underside of a breast of the subject such that the pericardial electrodes are correctly positioned for collecting the ECG data, receiving data from one or more of the precordial electrodes at a data collection device ECG measurement, and transmitting the ECG measurement data to a remote server from the data collection device.

Optionally, ECG measurement data is transmitted to the remote server from the data collection device via a user device.

Optionally, positioning the concave side the substrate around the underside of the breast of the subject includes supporting the breast over a comfort lip of the ECG lead system.

### BRIEF DESCRIPTION OF THE DRAWINGS

FIG. 1 is a schematic diagram of a diagnostic system in accordance with the principles of the present disclosure;

FIG. 2A is a rear view of an example ECG lead system of the diagnostic system of FIG. 1;

FIG. 2B is a front view of an example electrode patch of the ECG lead system of FIG. 2A;

FIG. 2C is a side view thereof;

FIG. 3A is a perspective view of a another example electrode patch including a comfort lip;

FIG. 3B illustrates the electrode patch of FIG. 3B with a hand of a user inserted therein;

FIG. 3C is a view of the electrode patch of FIG. 3A placed under the breast of a female patient;

FIG. 3D is a perspective view of another example electrode patch with a discontinuous comfort lip;

FIG. 4A is a perspective view of another example electrode patch;

FIG. 4B illustrates the electrode patch of FIG. 4A with fingers inserted therein;

FIG. 5A is a perspective view of another example electrode patch;

FIG. 5B illustrates the electrode patch of FIG. 5A in a folded configuration;

FIG. 6 is a perspective view of another example electrode patch;

FIG. 7 is a rear view of another example ECG lead system; and

FIG. 8 illustrates example components of computing devices that may be used for by or as various entities of the system.

### DETAILED DESCRIPTION

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” (or “comprises”) means “including (or includes), but not limited to.” When used in this document, the term “exemplary” is intended to mean “by way of example” and is not intended to indicate that a particular exemplary item is preferred or required.

In this document, when terms such “first” and “second” are used to modify a noun, such use is simply intended to distinguish one item from another, and such use is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

Additional terms that are relevant to this disclosure will be defined at the end of this Detailed Description section.

In the various embodiments, the devices, methods, and systems of the present disclosure provide a diagnostic system for measuring the physiological condition of a subject. Specifically, the diagnostic system includes an electrode patch that is configured for mobile ECG monitoring and that aids in correct placement of multiple electrodes on a subject. Embodiments of the current disclosure describe a diagnostic system including devices and methods used to record and create ECGs accurately, and with or without professional help. The present disclosure further includes a method of sensing, analyzing and/or transmitting or relaying a physiological signal.

The term “electrocardiography” process of producing an electrocardiogram (ECG) and is used in its broadest sense, including all methods for recording the electric activity of the myocardium and detecting the transmission of the cardiac impulse through the conductive tissues of the muscle, regardless of if this is performed in a clinical setting, to detect a malfunctioning of the heart, or in an athletic or private setting, monitoring the normal functioning of the heart. In the current disclosure, electrocardiography is performed using the devices disclosed herein.

It should be noted that while the systems and/or methods described herein refer to example implementations for a human subject, the disclosure is not intended to be limited to human subjects. In some implementations, the systems and methods described herein may be applied to and/or configured for any living subject. By way of a non-limiting example, the systems and methods herein may be applied to and/or configured for animal subjects.

As discussed above, traditionally twelve leads and ten electrodes are used when recording an ECG. An ECG is a graphical description of the electrical activity of the heart and is created by analyzing measurements obtained from several electrodes. An electrode is a conductive pad that is attached to the skin that enables recording of electrical currents. These electrodes send the recorded measurements or electrical signals to the ECG processing device (e.g., a processor) through leads. A lead may carry signals from one or more electrodes i.e., a measurement obtained from one (unipolar lead) or two electrodes (bipolar lead).

The electrodes used in a traditional ten electrode system include limb electrodes which can be placed on each of the subjects' limbs, and chest (or precordial) electrodes (V1-V6). The chest electrodes include: V1 which is placed at a fourth intercostal space at the right of the subject's sternum, V2 is at the fourth intercostal space to the left of the subject's sternum, V4 is placed at the fifth intercostal space at the midclavicular line, V3 is placed between V2 and V4, V5 is placed at the subject's anterior axillary line at the same level as V4, and V6 is placed at the subjects midaxillary line at the same level as V4 and V5. It is well known in the art that each of these electrodes is connected via a wire or cable to a signal processing unit where the electrocardiographic signals from each of these electrodes are translated into each of the twelve ECG leads. An ‘EKG lead’ refers to one of the 12 individual tracings on the standard EKG recording. The recording is the magnitude of the voltage (both + and −) as it changes with time during the cardiac cycle. The 12 EKG leads are I, II, III (bipolar), aVR, aVL, and aVF (unipolar)—all generated from the limb electrodes—plus V1, V2, V3, V4, V5, & V6 (unipolar). These electrodes must be individually (correctly) placed and subsequently connected to individual cables, a step which is both time consuming and prone to error. Proper lead placement requires not only proper electrode placement, but also attachment of the correct lead to each electrode. Errors in placement of electrode and/or leads can be anywhere from reversing limb leads to reversing chest leads. The most common errors are from misplacement of electrodes V1 and V2 (e.g., various studies have shown that up to 50% of errors can be due to misplacement of the V1 electrode). Similarly, other chest leads are also difficult to place and can vary depending on the individual is placing the electrodes leading to inconsistent readings and ECGs.

When recording an ECG, correct anatomical placement of electrodes is critically important to record an accurate signal because electrode placement directly affects the accuracy of electrical signals that are recorded from the heart as the heart muscle contracts and relaxes. The precise placement can be difficult even for medical professionals. If electrodes are inaccurately positioned, it can affect clinical interpretation of ECGs and the ECG can be inaccurate and/or incomplete. In some cases, errors in the ECG can lead to inaccurate or missed diagnosis of a subject and indicate that a subject's heart has issues such as arrhythmia, coronary heart disease, heart attacks, cardiomyopathy, or other issues that may not exist.

Traditional twelve lead/ten electrode placements further increase complications related to electrode placement and challenges in ensuring reproducible placement. Often, it is unnecessary to obtain ECG signals from each of the ten ECG electrode locations for a standard twelve lead ECG. The reduction in the number of leads is possible due to redundancy in the cardiac information present in each of the standard ECG leads. Therefore, a reduced electrode set comprising fewer than ten electrocardiographic electrodes may be used to directly obtain some of the ECG leads, while the information from the other ECG leads may be derived from the electrocardiograma signals obtained from the attached electrodes.

The current disclosure describes an electrode patch that includes pre-positioned and pre-connected precordial electrodes that allow for accurate placement of the precordial ECG electrodes on a subject without the need of a trained clinician. Additionally, in some embodiments, the patch may include fewer than six precordial electrodes that still provide results similar to a traditional twelve ECG lead arrangement. Broadly, the present disclosure relates to a diagnostic system which can detect electrical activity in a subject's heart and record ECGs accurately and consistently. Additionally, and/or alternatively, the devices and methods of the present disclosure allow for recording of the ECG to occur at home without the costs typically associated with a visit to a medical professional. In one aspect, the present disclosure relates to a system discussed in Published United States Patent Application US 2019/0269344A1 (the '344 Publication), the disclosure of which is incorporated herein in its entirety, and the ECG measurements and other information collected using the ECG system of the device can be processed and/or used as described in the '344 Publication.

While an exemplary embodiment of measuring ECG data is described below, it should be noted that, the current disclosure may be applied to the measurement of any physiologic data. For example, the disclosed systems and methods may be used to measure signals indicative of heart rate, activity level (e.g., physical mobility or movement), respiration rate, blood pressure (e.g., systolic and/or diastolic), blood oxygen saturation (SpO2), blood glucose or insulin level, pulse oximetry, impedance, body temperature, etc.

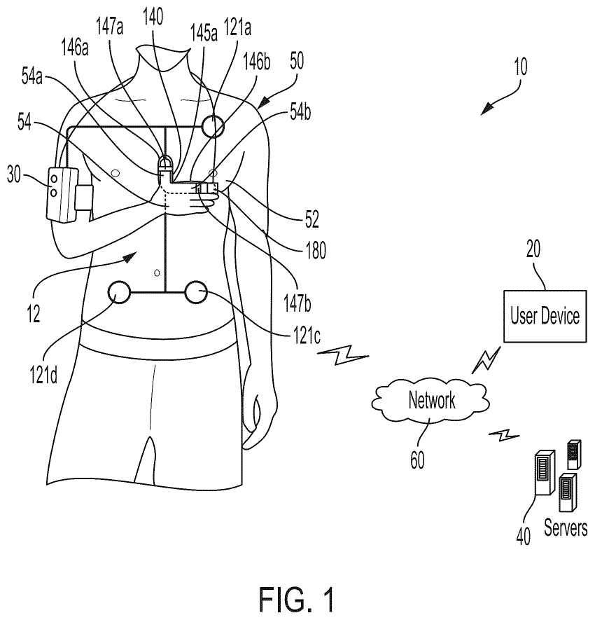

With reference to FIG. 1, a schematic representation of a diagnostic system 10 for measuring ECG of a subject according to the principles of the present disclosure is illustrated. The diagnostic system 10 includes an ECG lead system 12. In FIG. 1, a subject 50 has an ECG lead system 12 placed upon him or her, and attached by adhesive or other means, in order to monitor the physiological signals from the subject's heart (and/or for collecting other sensor data). The ECG lead system includes electrodes (as discussed below) configured to detect (and in some cases amplify) tiny electrical changes on the skin that are caused when heart muscles depolarize during each heartbeat. At rest, each heart muscle cell has a negative charge (called the membrane potential) across its cell membrane. Decreasing this negative charge toward zero, via the influx of the positive cations (Na+ and Ca++) is called depolarization. Depolarization activates mechanisms in the cell that cause it to contract. During each heartbeat, a healthy heart will have an orderly progression of a wave of depolarization that is triggered by the cells in the sinoatrial node, spreads out through the atrium, passes through the atrioventricular node and then spreads all over the ventricles. The depolarization wave (or ECG data) is indicative of the overall rhythm of the heart and is detected as variations in voltage between the electrode pairs.

The diagnostic system 10 also includes a user device 20, a data collection device 30, and a remote server 40 in communication with each other and/or the ECG lead system 12, via a network 60. The network 60 may be any now or hereafter known communication link or communication network and may utilize any now or hereafter know communication protocols. For example, in some implementations, wireless communication network may include one or more communication protocols such as, Bluetooth, Bluetooth Low Energy (BLE), radio frequency, WIFI, NFC, WLAN, ZigBee, and/or other communication protocols. Optionally, the data collection device 30 may be configured to be positioned or attached to the subject (e.g., via an arm band that receives the data collection device 30).

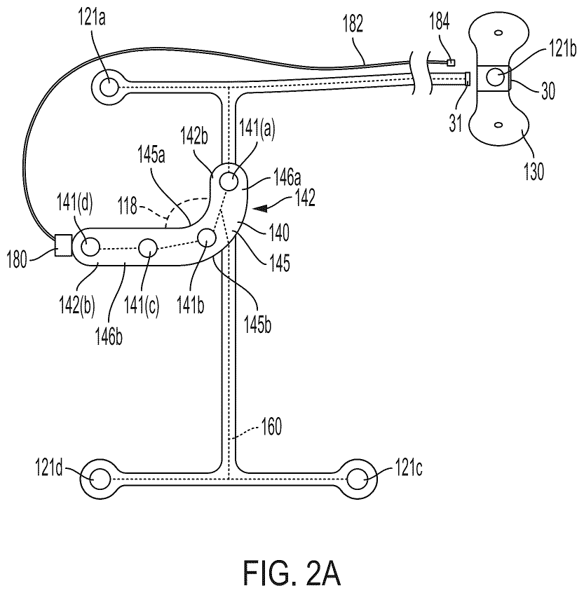

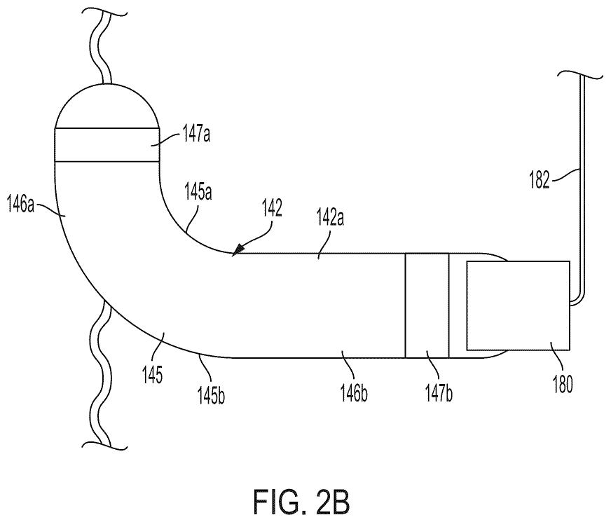

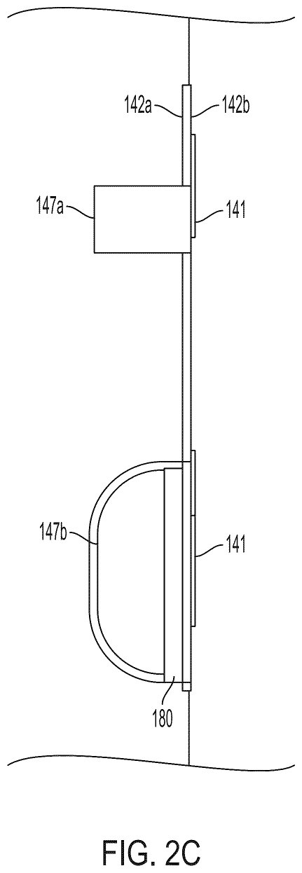

The diagnostic system may also include one or more sensors in communication with the user device 20, the data collection device 30, and/or the remote server 40 either directly and/or via the ECG lead system 12. Examples of sensors may include, without limitation, pulse oximeter, temperature sensor, heart rate sensor, blood pressure monitor, blood oxygen sensors, accelerometers, or the any other suitable sensor. For example, FIGS. 1 and 2A-2C illustrate a pulse oximeter 180 coupled to the electrode patch 140 of the ECG lead system 12 and positioned to receive the index finger 54B of the subject 50.

In various implementations, the ECG lead system 12 may include multiple electrodes configured to provide signals conveying information associated with the subject's ECGs. Data collection device 30 may be configured to receive and record information associated with ECGs. Information associated with ECGs may be communicated from the ECG lead system 12 to data collection device 30 through, for example, a wired connector (31 shown in FIG. 2A) and/or wirelessly such as via Bluetooth, NFC, etc. or any other suitable data transmission method. One or more of the electrodes of the ECG lead system 12 may include a transceiver to transmit the measured ECG data.

Data collection device 30 may be any type of portable electronic device known in the art. Data collection device 30 may include integrated circuits (microprocessor, memory, communication devices, etc.), visual displays (LED, LCD, etc.), and/or user interfaces (e.g., buttons) that can be activated by the patient 50. The integrated circuits of data collection device 30 may enable processing of collected ECG data, and communication between data collection device 30, the intermediate device (if any), and the remote server 40. The user interfaces (c . . . g, buttons) may enable the patient 50 to trigger an activity (data collection, communication with remote server 40, etc.) when the patient 50 feels uncomfortable (e.g., experiences chest pains, etc.), and the display may enable the data collection device 30 and remote server 40 to communicate with patient 50 (e.g., using text messages).

Data collection device 30 may be a portable device, sized and adapted to be kept in the possession (strapped, attached, placed in the pocket, etc.) of patient 50. Such a portable data collection device 30 may enable the patient 50 to go about the patient's daily activities while the data collection device 30 records (and/or transfers) ECG data. In the example embodiment illustrated in FIG. 1, data collection device 30 is shown as a device attached (e.g., strapped) to the patient's arm. However, this is only exemplary, and other configurations are possible. For example, in some embodiments, data collection device 30 may be combined with, or incorporated into a cellular phone (or another device, such as, smartwatch, etc.). In embodiments where the ECG lead system 12 is connected by a wire to the data collection device 30, data collection device 30 may include a connector to receive the connecting wire. In embodiments where ECG lead system 12 is coupled wirelessly, data collection device 30 may include a transceiver to communicate with a transceiver of ECG lead system 12.

Referring again to FIG. 1 and FIG. 2A, the band or strap 130 for holding the data collection device 30 and attaching it to a subject's arm is shown. The band 130 includes a band electrode which is the RA electrode 121b. The band 130 can additionally carry additional sensors (e.g., temperature sensors, accelerometers, etc.) In some embodiments, other sensors are disposed in other locations. For example, a pulse oximeter 180 is depicted as being coupled to the electrode patch 140 of the ECG lead system 12 and is positioned to receive the index finger 54B of the subject 50. The pulse oximeter 180 is connected to the data collection device 30 in a manner similar to the electrodes. As depicted, the pulse oximeter includes a wire 182 that connects to the data collection device 30 via a connector 184. In some other embodiments, the pulse oximeter and electrodes can share an electrical pathway to the data collection device 30.

In one or more embodiments, the data collection device 30 may include a display that configured to provide various information to a subject. Examples of such information may include, without limitation, status of the ECG device system (e.g., data collection device being ON, data collection being device OFF, ECG data being recorded or in progress), ECG data recording complete, etc.), date and time of recording, status of connection to the user device and/or the remoter server (e.g., connection active, connection terminated, instructions for establishing a connection, etc.), instructions for the subject (e.g., directions for proper electrode positioning, instructions to confirm or change electrode position, noisy signal, ECG recording instructions, etc.), sensor data (e.g., respiratory rate, oxygen level, temperature, etc. of the subject), or the like. Additionally, and/or alternatively, the data collection device may include other types of output device such as, without limitation, a speaker, a vibratory device, etc.

In some implementations, the measured ECG data and/or metadata/information associated with measured ECGs may be transmitted to remote server 40 from the data collection device 30 and/or directly from the ECG lead system 12. In some implementations, the transmission of information associated with ECGs to the remote server 40 may be performed wirelessly over network 60, via a cord, via optical signaling, by a portable storage medium (e.g., a USB drive), and/or via other methods. In some implementations, the data collection device 30 and/or the ECG lead system 12 may transfer the data to remote server 40 through an intermediate device (e.g., a user device 20). That is, the ECG data may be first sent to an intermediate device such as a user device 20 using Bluetooth or other similar technologies (RF, IrDA, NFC etc.), when data collection device 30 is in close proximity to the user device 20. The ECG data from the user device 20 may then be sent to the remote server 40 via, for example, the network 60. In Any of the data collection device 30, the user device 20, and/or the remote server 40 may be configured to receive, process, display, analyze, store, print, wired or wireless transmit, and/or otherwise utilize the measured ECG data and/or metadata/information associated with a measured ECGs. Optionally, a physician can review the data collected (e.g., via the data collection device 30, the user device 20, and/or the remote server 40) and provide the subject 50 with treatment recommendations.

In use, the subject 50 will power on the data collection device 30. Once powered on, the subject 50 connect the data collection device 30 to a personal device (e.g., user device 20). In some embodiments, the subject will be prompted or follow instructions to correctly position the electrodes of the ECG lead system 12. The subject will then place the signal acquisition box to their arm and connect the ECG lead system 12 to the data collection device 30. The data collection device 30 can begin to collect data from the ECG lead system, if the electrodes of the ECG lead system are placed incorrectly, the data collection device 30 is able to communicate with the personal device of the subject and inform the subject to place the electrodes in different locations in order to record an accurate ECG. Once the electrodes are accurately placed, or alternatively before the electrodes are accurately placed, any additional sensors being used can be connected to the data collection device 30. After all sensors are placed, the data collection device can begin to record data from the electrodes and sensors. The recorded data is then be uploaded to the remote server either by the personal device or by the data collection device. The subject can receive a recommendation for medical treatment via the personal device or have a meeting with a physician immediately.

Optionally, the data collection device may include a power source (e.g., a rechargeable battery) for providing power to one or more components of the ECG lead system 12 and/or other sensors of the diagnostic system.

Referring now to FIGS. 2A-2C, an example ECG lead system 12 including an electrode patch 140 is illustrated. As shown in FIG. 2A, the ECG lead system 12 includes three or four limb electrodes 121 (a)-(d) (Right Arm (RA), Left Arm (LA), Left Leg (LL) and Right Leg (RL) electrodes, respectively) configured for placement on or near the corresponding limb. Generally, the LA electrode can be placed anywhere between the left shoulder and the left wrist or below the left clavicle of the subject, the RA electrode can be placed anywhere between the right shoulder and the right wrist or below the right clavicle of the patient, the LL electrode can be placed anywhere between the left ankle and left side of the waist of the patient or at the upper left abdominal quadrant, and the RL electrode can be placed anywhere between the right ankle and right side of the waist of the patient or at the upper right. abdominal quadrant. For example, as shown in FIG. 1, the LA electrode may be positioned below the clavicle of the subject (e.g., as close to the left shoulder as possible), the RA electrode is positioned below the band of the data collection device 30 (discussed in further detail later), the LL and RL electrodes are placed at the left and right upper abdominal quadrants respectively. The ECG lead system 12 also includes an electrode patch 140 including at least four precordial electrodes 141 (a)-(d) (optionally, six electrodes) in electronic communication with the limb electrodes (e.g., via electrode wires or leads).

In some aspects, the present disclosure relates to an electrode patch including four precordial electrodes (instead of six) that can record ECGs with the accuracy of the traditional ten electrode and twelve lead electrode system described above. Specifically, V1 and V2 both assess the interventricular septum, and incorrect placement of V1 is a known cause of ECG errors. As such, in some aspects of the current disclosure, the electrode patch does not include the V1 precordial electrode. Additionally, V6 often overlaps with the LL limb electrode rendering it redundant, and in some aspects of the current disclosure, the electrode patch does not include the V6 precordial electrode. In some embodiments, the present disclosure describes an ECG lead system 12 that includes four limb electrodes and an electrode patch including precordial electrodes V2-V5. Optionally, the electrode patch 140 may be extendable via one or more peripheral extenders (discussed below) to include peripheral precordial electrodes V1 and V6.

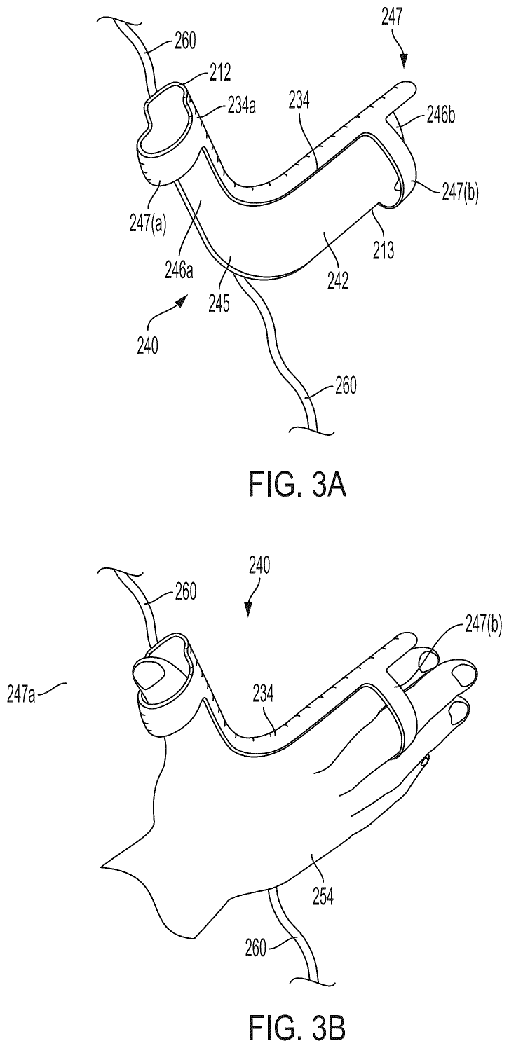

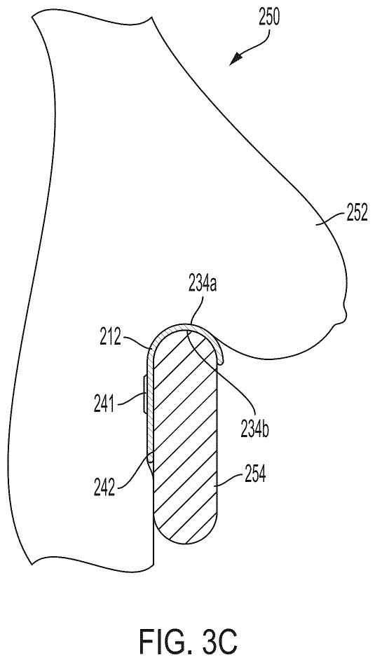

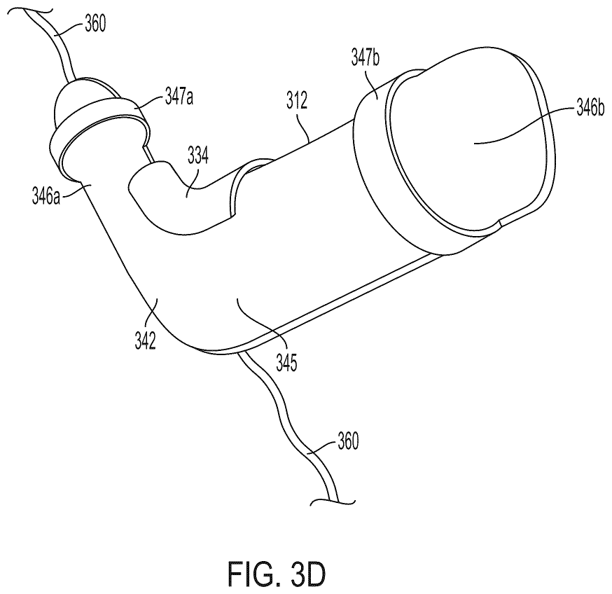

In various embodiments, the electrode patch 140 is designed in a shape for aiding in correct placement of the same (as discussed below), and the electrodes 141 (a)-(d) may be positioned on electrode patch 140 to align with one or more desired anatomical locations on a human subject when the electrode patch 140 is correctly applied or positioned. As such, responsive to a user applying the electrode patch 140 to a human subject, a majority of the electrodes may be located in the proper position for performing an ECG. In some embodiments, the electrode patch 140 includes a central portion 145 flanked by two peripheral portion 146a and 146b, where the central portion 145 includes an inner (breast-facing) concave side 145a (i.e., a side or an inner edge that is concavely curved) that when positioned under the left breast 52 of the subject 50 will follow the curvature of the breast and naturally place the electrodes in correct positions. Optionally, the concave edge may form an inner curved surface or lip (as discussed below with respect to FIGS. 3A-3C). In some embodiments, the concave edge 145a is sized (e.g., has a radius of curvature, arc angle, curvature, and/or an arc length) to extend at least partially around the breast of the subject. For example, the radius of curvature the concave edge 145a may be chosen such that the concave edge 145a can approximately concentrically align with an underside of the breast of the subject (i.e., is approximately equal to or slightly greater than the radius of curvature of the underside of the breast). In some embodiments, the radius of curvature may be chosen based on the average radius of curvature of a breast of a subject of certain size, gender, etc. Optionally, the radius of curvature of the concave edge 145a may be approximately the same size as an average radius of curvature of the webspace or contour between an index finger and a thumb of a subject of certain size, gender, etc. (for case of positioning using the index finger and thumb, as discussed below). In some embodiments, the radius of curvature of the concave edge 145a can be about 2 inches, about 3 inches, about 4 inches, about 5 inches, or about 6 inches or any other suitable radius of curvature. In some embodiments, the radius of curvature can be between about 2 inches-6 inches, about 3 inches-5 inches, about 3.5 inches-4.5 inches or any other suitable range. The arc length of the concave edge 145a may be about 2 inches, about 2.5 inches, about 3 inches, about 3.5 inches, about 4 inches or any other suitable length. In some embodiments, the arc length is between about 2.5 inches-4 inches, about 3 inches-3.5 inches or any other suitable range. The arc angle 118 may be chosen such that the concave edge 145a can approximately concentrically align with an underside of the breast of the subject and/or is similar to an angle between a thumb and in index finger of a subject's right hand. In some embodiments, the arc angle is configured to facilitate accurate placement of the electrode patch. In some embodiments, the arc angle 118 is about 80 degrees, approximately 90 degrees, about 100 degrees, or about 110 degrees. In some embodiments, the arc angle 118 is in a range between 90 degrees-110 degrees, 95 degrees-105 degrees, or any other suitable range.

Further, the electrode patch 140 may be dimensioned and shaped such that the subject's fingers (e.g., the index finger, index finger and middle finger, all four fingers, etc.) and thumb can align at least partially with the two peripheral portions (146a and 146b) in order for the contour between the index finger and the thumb to, at least partially, align with, and position the concave edge 145a under the left breast. Optionally, the lengths of the two peripheral portions 146a and 146b may be chosen to be approximately similar to the lengths of the thumb and fingers, respectively of a subject's right hand (e.g., the average lengths for an index finger and a thumb of a subject of certain size, gender, etc.). In some embodiments, the peripheral portion 146b is sized to have a length that is at least as long as the size of an index finger of a subject. In some other embodiments, the peripheral portion 146b is sized to have a length that is at least as long as the size of another finger (e.g., middle finger, ring finger, pinky finger, average size of two or more fingers, etc.) of a subject. While the disclosure describes the use of index finger for holding the peripheral portion, it is not so limiting, and another finger or combination of two or more fingers may be used; and the peripheral portion 146b may be sized accordingly. In some embodiments, the peripheral portion 146a is sized to have a length that is at least as long as the size of the thumb of a subject. In some embodiments, the lengths of the peripheral portions 146a and/or 146b can be shorter than the length of the thumb and finger(s) of a subject such that the tip of finger(s) (e.g., index finger) or the thumb can extend outwards of the electrode patch and attach to a sensor (e.g., a pulse oximeter 180 which takes measurements from the tip of a finger, or another sensor). In some embodiments, the length of the peripheral portion 146a is about 1 inch, about 2 inches, about 3 inches or any other suitable length. In some embodiments, the length of the peripheral portion 146a is between about 1 inch-3 inches, about 1.5 inches-2.5 inches about 2 inches-3 inches or any other suitable range of lengths. In some embodiments, the peripheral portion 146b has a length of about 3 inches, about 4 inches, about 5 inches, about 6 inches or any other suitable length. In some embodiments, the peripheral portion 146b has a length of between about 3 inches-6 inches, about 4 inches-5 inches, or any other suitable range of lengths.

In some embodiments, the electrode patch 140 is shaped approximately to mimic to the natural L-shape of a thumb and index finger of a right hand of a subject when the index finger and thumb are opened, also known as the purlicue web space. For example, in FIG. 1, the thumb 54A and the index finger 54B of the left hand of the subject 50 are open to align with the peripheral portions 146a and 146b such that when the hand 54 is positioned below the breast 52 (e.g., to cup the breast 52 from the underside), the concave edge 145a partially surrounds the underside of the left breast 52 to correctly position the electrodes on the electrode patch 140. In some embodiments, the two peripheral portions (146a and 146b) are straight portions that extend from either side of a central portion 145. In some other embodiments, the two peripheral portions (146a and 146b) can be configured as curvilinear or shaped otherwise to conform to the subject's thumb and index finger. It will be appreciated that in other embodiments, the two peripheral portions (146a and 146b) extend from the curved portion and are separate pieces. Other shapes of the electrode patch are additionally possible, for example, hand shaped, curvilinear, or any other suitable shape.

It should be noted that an outer surface 145b of the central portion 145 may have any shape. For example, in the embodiments of FIGS. 2A-2C, the outer surface is also concavely curved and has a larger radius of curvature compared to that of the inner concave edge 145a. In another embodiment, the outer surface is also concavely curved and has a smaller radius of curvature compared to that of the inner surface. In other embodiments, the outer surface may be concavely curved and may have the same radius of curvature as that of the inner surface. Alternatively, the outer surface may assume another shape such as, without limitation, a surface formed by two straight lines intersecting at an angle (e.g., at about 90°, at about 80°, etc.).

Furthermore, the patch may be provided in different sizes suitably dimensioned for subjects of various sizes (e.g., based on the size of the index finger/thumb of the right hand of a subject and/or the radius of curvature of the underside of the left breast of a subject). Furthermore, owing to anatomical differences between men and women, different electrode patch sizes are preferably supplied to men and women.

Optionally, to facilitate positioning of the concave edge 145a under the left breast of a subject, the patch may include certain markings and/or structural features indicating one or more locations of the patch that a subject should align with a landmark on the subject's body. The patch may also carry printed instructions in the form of pictures, e.g., a schematic instruction showing where the patch should be placed, an arrow, a pictogram or text explaining how the patch should be placed.

Referring back to FIGS. 1 and 2A-2C, the electrode patch 140 includes a substrate 142 having an upper surface 142a (i.e., non-skin facing) and a lower or base surface 142b (i.e., skin facing surface), where the lower surface 142b includes the electrodes 141 (a)-(d) (V2-V5) for sensing a physiological signal from a subject. Optionally, at least certain portions of the upper surface 142a may be a hand contacting surface that contacts at least a portion of the hand of a subject while holding the electrode patch against the chest (or precordium of the subject). The electrode patch further comprises one or more electronic components (not shown here) such as, without limitation, transmitters, a DC-power source (e.g., a battery), transmitters, switches, signal filters, amplifiers, antennas, or any other suitable component. The one or more electronic components may comprise one processing chip, multiple chips, single function components or combinations thereof, which can perform all of the necessary functions of detecting the physiological signal from the electrodes, transmitting a signal corresponding to the physiological signal to a receiving unit (e.g, the data collection device) and optionally receiving a signal from a remote transmitter. These one or more electronic components can be assembled on a printed circuit board or by any other means known to those skilled in the art.

Specifically, the one or more electronic components are used to receive the physiological signal from the electrodes. The one or more electronic components also transmit or store a signal corresponding to the physiological signal to the data collection device 30. In a number of embodiments, the electrode patch further comprises electrical pathways 160 connecting the electrodes to one or more electronic components for receiving the physiological signal. The electrical pathways may be attached to the substrate. For example, the electrical pathways 160 are each a line or trace of conductive ink (or coating material), which is printed on the upper and/or lower surface of the substrate. Optionally, any of the electrodes, electronic components and their electrical connections can be printed on the substrate of the electrode patch (e.g., using screen printing). Other types of electronic components and electric connections attachments (e.g., mechanical attachments) are within the scope of this disclosure. As depicted in FIG. 2A, the electrical pathway 160 can connect to the data collection device 30 via a mechanical connector 31. In some embodiments, electrical pathways 160 may include conductive traces that extend from the electrodes of the patch to terminals (not shown here) of the electrode patch. These traces may include any conductive material and may be provided by any known additive and/or subtractive processes (masking and plating/deposition, plating/depositing and etching, etc.). These traces may extend along the lower skin-facing surface or the exposed surface of the electrode patch. The conductive traces may direct the measured physiological signals from electrodes to the terminals. The terminals may be the ends of traces positioned on the upper surface of the substrate. In embodiments where the traces extend along the lower surface, a through-hole via may electrically connect ends of the traces to the terminals. In general, the terminals may be positioned anywhere on patch. In some embodiments, the terminals may be positioned on an edge of the substrate 142. In such embodiments, an external wire (e.g., 182) attached to (e.g., clipped, etc.) the terminals may retrieve the measured signals from the electrode patch. In some such embodiments, the data collection device may receive the measured signals from terminals via the wire 182 (and/or wirelessly). In some embodiments, electrode patch may also include circuitry (not shown) to process (amplify, filter, etc.) the physiological signals measured by the electrodes.

The individual ones of the multiple electrodes may be configured to provide signals conveying information associated with ECGs. The electrodes can be any type of electrode known to those skilled in the art for sensing a physiological signal. Preferably, the electrodes of the present invention can be conventional electrodes known to those skilled in the art comprising a sensing element and a conductive gel for transmitting the signal between the subject's skin and the sensing element; or dry electrodes comprising a penetrator for detecting physiological signals below the surface of the skin as a sensing element. The electrodes don't have to be of the same type, i.e., for example one could be a conductive gel electrode while the others are dry electrodes. The electrodes can be any shape known to be useful to those skilled in the art. For example, the electrodes can be circular or non-circular in shape. Conductive gel may be a viscous conductive medium that fills any gap between the electrodes and the patient's skin to improve detectability of electrical activity under the skin. Any type of commercial or specially formulated gel known in the art may be used as conductive gel.

With continued reference to FIG. 1 and FIGS. 2A-2C, the electrodes 141a-d are mounted to the substrate 142 at positions that correspond with electrode placements V2-V5 on the chest of a subject for recording an accurate ECG, when the electrode patch 140 is correctly positioned using the concave edge 145a. In some embodiments, the specific locations of the electrodes (i.e., the spacing between the electrodes) vary with the size of the electrode patch, where the size of the electrode patch is chosen based on the size, gender, etc. of the subject (as described above). In some embodiments, the center-to-center distance between electrode 141a and 142c, is about 2 inches, about 3 inches, about 4 inches, about 5 inches or any other suitable distance. In some embodiments, the distance is between about 2 inches-5 inches, about 3 inches-4 inches, or any other suitable range of distances. In some embodiments, electrode 141b is approximately centered between electrodes 141a and 141c. In some embodiments, there is a center-to-center distance between electrode 141c and 141d corresponding to accurate placement of electrodes V4 and V5. In some embodiments, the distance, between electrode 141c and 141d is about 2 inches, about 3 inches, about 4 inches, 5 inches, or any other suitable distance. In some embodiments, the distance is between about is about 2 inches-5 inches, about 3 inches-4 inches, or any other suitable range of distances.

It should be noted, the electrode patch 140 can include additional electrodes and correspond to a traditional ten electrode twelve lead ECG system or any other contemplated ECG system. Optionally, the ends of the peripheral portions 146a and/or 146b may include coupling (e.g., Velcro, adhesive based, tabs, etc.) means for attachment of additional electrodes (e.g., corresponding to V1 and/or V6) or substrates including additional electrodes to the electrode patch.

The electrode patch is attached to the subject by any method or means known to those skilled in the art. By way of example but not limitation, the electrode patch may be attached to the subject using adhesive on the lower surface (i.e., skin facing side) of the substrate, using adhesive on the electrodes on the lower surface of the substrate, by an elastomeric band that is attached to the substrate and about the subject, using tape, using sutures, using clips, or some combination thereof. If an adhesive is used to attach the electrode patch to the subject, preferably the adhesive is biologically compatible to the subject. The adhesive may include a material that allows the substrate to be easily removed and reattached, if desired. The adhesive also may include a material that is safe and non-irritating for the patient's skin. More preferably, a pressure sensitive adhesive is used. Even more preferably, a removable pressure sensitive adhesive is used. The adhesives used include but are not limited to for example natural rubber, butyl, styrene block copolymer, SBR, acrylics, hydrogels, polyurethanes, hydrocolloids, and silicone-based adhesives. Optionally, there are provided on each of the electrodes an electrically conductive, adhesive gel (not shown) which may, if desired, be covered with a wax liner or other removable protective material that may be peeled off before use. The gel may be specifically formulated to adhere to the patient's skin and to provide good electrical connection whilst allowing painless removal after use. For example, the gel may be a hypo-allergenic, silver/silver-chloride gel.

The substrate 142 having the upper and the lower surface can be made from any materials known to those skilled in the art. Preferably the substrate 142 is made from a material which has the mechanical features necessary for attachment of the electrodes and for attaching to the one or more electronic components. Optionally, the substrate is made from a material that allows a certain flexibility that is adapted to conform to the contours of the patient's skin, depth necessary for wells or depressions to hold conductive electrode gels or pastes, and/or that prevents (or reduces) absorption of biological contaminants. In some embodiments, the substrate may have pores (or otherwise breathable) or other features to enhance patient comfort. Examples of the substrate material include, without limitation, a silicone-based material, fabric, a thermoplastic elastomer, a polyurethane foam, closed cell foam or any other suitable material. The electrode patch 140 may be disposable or reusable.

In some embodiments, the substrate includes a material that provides a desired stretchability such that the substrate material may be stretched when a stretching force is applied to it and when the force is released, the substrate may return to its original state such that the electrodes are substantially in the same location relative to each other as was the case prior to stretching. In some embodiments, the substrate may lack such a memory such that upon stretching and thereafter removal of the stretching force, the substrate does not contract, and the electrodes remain in their stretched position. It should be appreciated that when the substrate is stretched and attached to the skin of the patient, the patient's skin will act to maintain the stretching force on the substrate with little to no contraction of the substrate.

In some embodiments, the electrode patch is configured to remain on the subject's chest for a desired period of time, that is, the desired period of ECG monitoring, without irritating the subject's skin. For example, in some embodiments, the electrode patch is configured to remain on the subject's chest for between about 15 minutes and about 1 hour. In other embodiments, the electrode patch is configured to remain on the subject's chest for up to about 12 hours, 24 hours, or 72 hours, depending upon the length of monitoring. In some embodiments, a silicone-based material forming the substrate may maintain the electrode patch on the subject's chest for the desired period of time, as described, while not irritating the skin. In some embodiments, the ECG monitoring takes place when the subject is laying down. In other embodiments, the ECG monitoring can take place when the subject stands.

In some embodiments, the upper surface 142a of the substrate 142 includes one or more structural elements. Optionally, the structural elements extend or orient away from the base surface of the substrate. In some embodiments, the structural elements are configured to assume a curvature or curved profile in an unloaded state.

In the example embodiment shown in FIG. 2A, the structural elements are one or more supports 147a-n (collectively, 147) which allow the subject to hold the electrode patch to the chest and/or as ECG measurements are being recorded. In some embodiments, the support 147 is a loop that is configured to extend around a finger(s) or a thumb of the subject. In some embodiments, the support 147 includes multiple loops configured to extend around multiple fingers of the subject. In other embodiments, the support 147 is a webbing configured to extend around a hand of the subject 50 (discussed in further detail below). It will be appreciated that any supports allowing for the subject comfortably hold the electrode patch in place are additionally possible.

For example, as shown in FIGS. 1 and 2A-2C, the first support 147a is a loop mounted on the first peripheral portion 146a, that is sized and configured to receive at least the thumb of a subject (for e.g., the diameter may be chosen based on the average thumb diameter of subjects of a certain size). The second support 147b is a loop mounted on the first peripheral portion 146b, that is sized and configured to receive at least the index finger of a subject (for e.g., the diameter may be chosen based on the average index finger diameter of subjects of a certain size). It will be appreciated that the first and second loops 147a and 147b can be configured to receive additional fingers. For example, the second loop 147b can be sized and configured receive the subject's index and middle finger or any other suitable combination of fingers. In a preferred example, as shown in FIG. 1 the loop 147a can receive the thumb 54A and the loop 147b can receive at least the index finger 54B of the subject 50, while the thumb 54A and the index finger 54B of the subject are opened such that the subject 50 can hold the electrode patch 140 comfortably for positioning on the chest and/or during ECG recording on the underside of the subject's left breast.

Optionally, in some embodiments, the first peripheral portion 146a and/or the second peripheral portion 146b may include structural elements comprising a blocking element configured to prevent the corresponding finger or thumb to slide over the edge of the corresponding peripheral portion. For example, the blocking element may be an approximately vertical extension positioned at or near the edge (away from the central portion) of the upper surface of the peripheral portion that prevent the corresponding thumb or finger from sliding over the edge of the peripheral portion. In another example, the blocking element may be a curved extension positioned at or near the edge (away from the central portion) of the upper surface of the peripheral portion that at least partially cups the tip of the corresponding finger(s) or thumb. For example, FIGS. 9 and 10 illustrate example embodiments including blocking element 448 is mounted on the first peripheral portion 446a, and is sized and configured to cup the tip of at least the thumb of a subject (for e.g., the diameter may be chosen based on the average thumb tip diameter of subjects of a certain size). The second support blocking element 447 is on the second peripheral portion 446b, that is sized and configured to receive the tip of at least the index finger of a subject (for e.g., the diameter may be chosen based on the average index finger tip diameter of subjects of a certain size). It will be appreciated that the first and second blocking elements 447a and 447b can be configured to receive additional fingers.

Referring to FIGS. 3A-3C, another example electrode patch 240 that is similar to the electrode patch 140 is depicted. The electrode patch 240 includes a structural element that is configured as lip portion that extends partially from an inner side or edge of the substrate 242 (i.e., the side of the substrate that faces the underside of a subject's breast) for ease of placement of the electrode patch at the underside of a subject's breast and for comfort of the subject. The lip is oriented away from the bottom surface of the substrate and naturally assumes a curvature/curved profile in an unloaded state. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

As shown in FIGS. 3A-3C, the inner side or edge of the substrate 242 of the electrode patch 240 includes or integrally forms a comfort lip 234. The comfort lip 234 extends from an inner edge 212 (e.g., the inner concave edge) defined by a central portion 245, and two peripheral portions 246a, 246b (similar to the central portion 145 and peripheral portions 146a, 146b discussed above). As depicted in FIGS. 3A-3C, the comfort lip is continuous along the side 212, however the comfort lip could be discontinuous (for example, see FIG. 3D) and only extend from only a portion of the side 212, for example, the comfort lip could only extend from the inner edge of one of the peripheral portions 246a, 246b; extend from the inner edge of both of the peripheral portions 246a, 246b; extend from the inner edge of only the central portion 245; or extend only from the inner edge of certain other portions of the substrate (as discussed below). The comfort lip 234 is configured to provide padding and support to the breast of the subject when the electrode patch is positioned below the breast. In some embodiments, the comfort lip is integral with a substrate 242 of the electrode patch 240. It will also be appreciated that the comfort lip 234 can be configured to be removable from the substrate 242. For example, the comfort lip 234 could be configured to attach or mount to an inner edge 212 of a substrate of an electrode patch via Velcro, zip, or another suitable attachment mechanism. For example, the comfort lip 234 could be configured to attach to a different substrate for example the substrate 142 of FIG. 2A.

As shown in FIGS. 3A and 3B, the substrate 242 also includes structural elements comprising supports 247a and 247b in the form of loops that extend between the comfort edge 234 and the outside edge 213 of the substrate 242. The loops 247a and 247b are configured to extend around a finger(s) or a thumb of the subject (as discussed above). For example, FIG. 3B illustrates a hand 254 of a subject holding the electrode patch 240 by inserting the thumb within the support loop 247a and two fingers within the support loop 247b. It will be appreciated that the loops 247 can be removable.

Referring to FIG. 3C, a side view of the electrode patch 240 is shown in use with a female subject 250. The comfort lip 234 is shown extending around a hand 254 of the female subject 250 when the electro patch 240 is placed below the breast 252 of the subject. In some embodiments, the comfort lip 234 is configured to extend around the hand 254 (for example, see FIG. 3B) of the subject so that the breast 252 of the subject 250 contacts the comfort lip 234 when the electrode patch 240 is positioned below the breast 252 of the subject instead of an edge of the electrode patch 240 (which might cause discomfort to the subject because of, for example, digging of the edge into the breast tissue). As such the comfort lip is configured to provide a comfortable place for the breast 252 of the subject 250 to rest on. Optionally, the comfort lip covers at least the portion of the hand of the subject to provide padding to the breast of the subject.

In some embodiments, the comfort lip includes a breast contacting portion and a hand contacting portion. In some embodiments, the breast contacting portion is configured to provide a comfortable place for the subject's breast to rest on and the hand contacting portion is configured to extend over the hand of the subject. Referring to FIGS. 3A-3C the comfort lip 234 is approximately U shaped and a convex portion 234a of the U shape extends under the subject's breast and is the breast contacting portion. A concave portion 234b of the U shape extends over the hand 254 of the subject 250 and is the hand contacting portion. In other embodiments, the comfort lip 243 can extend partially around the hand 254 of the subject 250 depending on the amount of the subject's breast 252 that extends over the comfort lip 234. In some embodiments, U-shape of the comfort lip 234 has a radius of curvature that is sized to fit over the hand 254 of the subject. In some embodiments, the radius can be about 0.2 inches, about 0.4 inches, about 0.6 inches, about 0.8 inches, about 1 inch, about 1.2 inches or any other suitable size of radius. In some embodiments, the radius is between about 0.2 inches-1.2 inches, about 0.4 inches-0.9 inches, about 0.5 inches-0.6 inches or any other suitable range of sizes. It will also be appreciated that the U shape may only be configured as a partial U shape and only partially extend over the patient's hand. For example, the U shape could be configured as any shape that secures the electrode patch 240 to the hand of the patient.

In some embodiments, the comfort lip 234 is formed from a soft material that is pliable and lacks rigidity such as a foam or fabric. In some embodiments, the comfort lip is made from a flexible material that is pliable and bends. In other embodiments, the comfort lip 234 is formed from a semi-flexible material that is pliable but retains the overall shape of the comfort lip after it has been bent. In some embodiments, the comfort lip 234 is formed from a webbing, in other embodiments the comfort lip is formed from a silicone rubber, a thermoplastic elastomer, a polyurethane foam, or any other suitable material. Alternatively, the comfort lip 234 may be formed from a semi-rigid material that supports the hand of a user without loops 248.

Referring to FIG. 3D, an example electrode patch 340 that is similar to the electrode patch 240, except for including a different comfort lip 334, is shown. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

The electrode patch 340 includes a substrate 342 and the comfort lip 334. The electrode patch 340 includes a central portion 345 flanked by two peripheral portions 346a, 346b flanking the central portion 345 (similar to the peripheral portions 146a, 146b discussed above).

The comfort lip 334 extends along an edge 312 (e.g., the concave edge) defined by a side of the central portion 345. As depicted, the comfort lip 334 only extends along a portion or subset of the edge 312. The comfort lip 334 is configured (i.e., located and/or sized) for creating and/or maintaining correct positioning and shape of the purlicue web space (i.e., the crutch of the hand) with respect to the underside of the subject's breast during application of the electrode patch 340. Specifically, in the absence of the comfort lip, if the finger loops (e.g., the loops 347a and/or 347 b) are not properly sized with respect to the fingers/thumb of the subject's hand, the purlicue web space may form a smaller radius of curvature (when the loops are too small with respect to the fingers/thumb of the subject's hand) or a larger radius of curvature (when the loops are too big with respect to the fingers/thumb of the subject's hand causing the fingers/thumb to extend too far out) with respect to the underside of the breast of the subject where the electrode patch needs to be positioned. Such changes in the radius of curvature of the subject's hand holding the electrode patch can cause incorrect positioning of the electrode patch (too far down on the chest when the purlicue web space forms a smaller radius of curvature or vice versa). As such, addition of a comfort lip that can guide the position of the purlicue web space, and thereby achieve a desired radius of curvature can be used for creating and/or maintaining correct positioning of the electrode patch 340 for ECG measurements.

The comfort lip 334 additionally assists to prevent accidental movement of the electrode patch 340. For example, the electrode patch 340 could slip out of position when held for a sustained amount of time if the comfort lip 334 was not present.

While FIG. 3D illustrates a single comfort lip 334 extending from the inner edge of the central portion 345, the disclosure is not limiting. Any desired number and/or placement of the comfort lip may be utilized as long as the comfort lip(s) can be used for proper positioning and radius of curvature (i.e., shape) of the purlicue web space (i.e., the crutch of the hand) with respect to the underside of the subject's breast during application of the electrode patch 340. For example, two comfort lips may be provided extending from at least parts of the inner edges of the peripheral portions 346a and 346. The comfort lip 334 could alternatively be configured as multiple discrete portions or a different configuration to prevent slippage or movement of the hand. It will also be appreciated that as the comfort lip 334 is positioned on the concave edge 312 of the central portion, it may also function to provide support and comfort to the patient's breast.

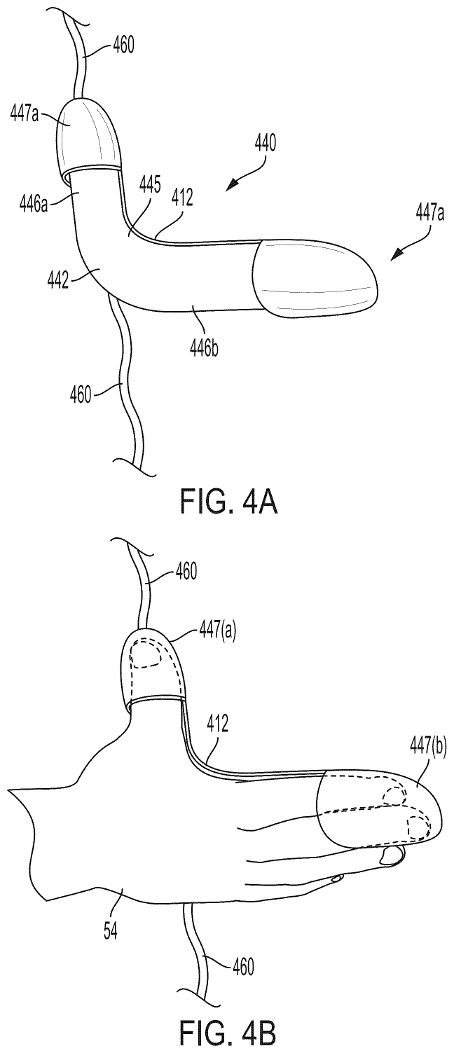

Referring to FIGS. 4A and 4B, an example electrode patch 440 that is similar to the electrode patch 140 but including different types of structural elements is depicted. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

The electrode patch includes a substrate 442 that carries electrodes (not shown). The electrode patch includes an inner edge 412 (e.g., a concave edge) that is shaped and configured to accurately position electrodes (not shown) on the subject similar to edges 212, 312. The electrode patch 440 additionally includes a central portion 445 flanked by two peripheral portions 446a, 446b (e.g., similar to the central portion 145 and peripheral portions 146a, 146b).

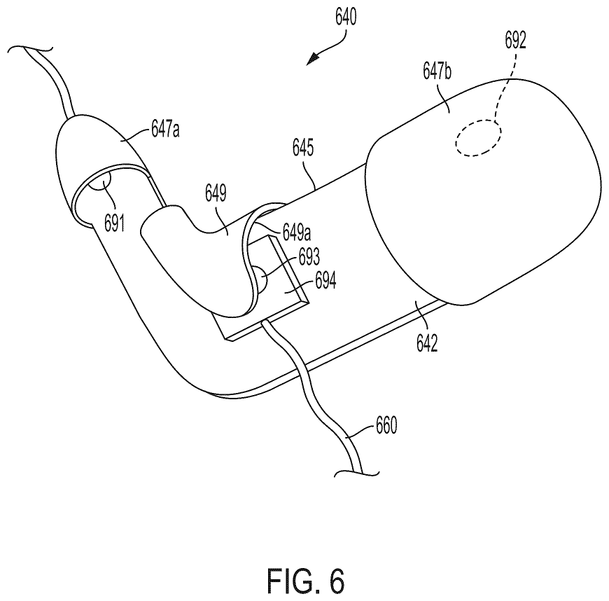

The support 447 of the electrode patch 440 includes structural elements comprising finger cups 447a and 447b (collectively, 447) that are configured to act as a barrier to prevent over extension of fingers holding the electrode patch for placement such that the radius of curvature of the purlicue web space becomes larger than that required for correct placement of the electrode patch at the underside of a subject's breast. Specifically, in the absence of a comfort lip or a barrier, the fingers/thumb of a subject may extend too far into or out the support 447 causing an undesirable increase in the radius of curvature of the purlicue web space. The finger cups 447a and 447b are configured to cup the tips of the fingers/thumb of the subject to prevent such over extension. As shown in FIG. 4B, the finger cup 447a is sized to receive a thumb (and snugly hold—e.g., like a glove) of the subject and the finger cup 447b is configured to receive two fingers (and snugly hold—e.g., like a glove) of the subject (or any desired number of fingers) when the hand 54 of the patient is opened. FIG. 6 illustrates an embodiment in which the finger cup 447b is sized and configured to encase all four fingers of a user's hand.

Similar to the embodiments discussed above, the electrode patch 440 may include various sizes of finger cups to fit different hand sizes and/or number of fingers. For example, the electrode patch may be sized as small, medium, large, and other suitable sizes. In some examples, the electrode patch 440 may be configured for males or females. It should also be noted that while FIGS. 4A and 4B illustrate finger cups, other types of barriers (e.g., a vertical plate) for preventing over extension of fingers/thumbs within or out of the finger loops or supports are within the scope of this disclosure.

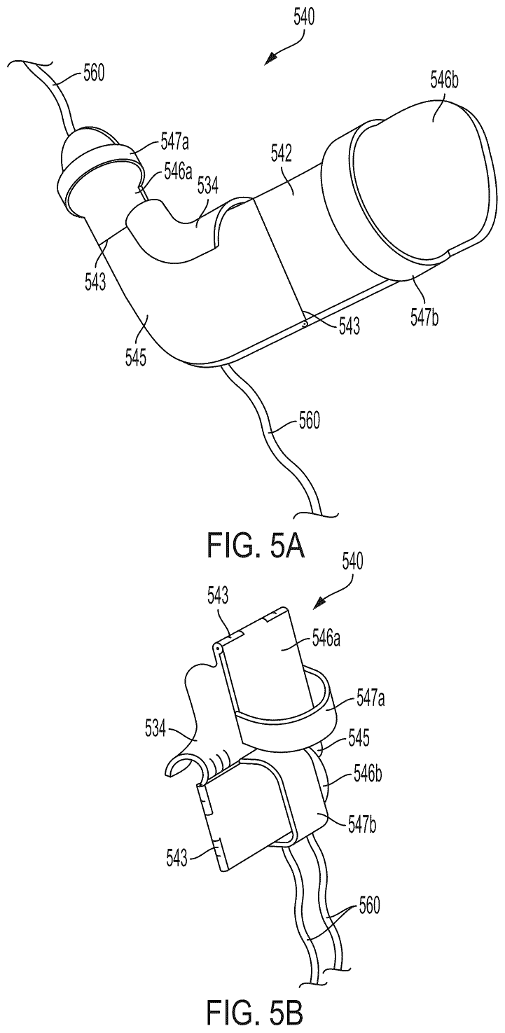

Referring to FIGS. 5A and 5B, an example electrode patch 540 that is similar to the electrode patch 340 but including a foldable substrate 542 is depicted. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

As shown in FIG. 5A, the substrate 542 may be a foldable substrate to allow for case of storage and/or transportation. In some embodiments, the patch may comprise a single substrate 542 with hinges 543. Alternatively and/or additionally, the substrate 542 may comprise a plurality of foldable substrate components that are connected together (e.g., via hinges provided at the fold lines 543) that may be folded onto (e.g., on top of) one another. Examples of hinges may include pinned hinges, living hinges, piano hinges, fold lines, or other similar hinges. For example, as shown in FIG. 5B, the peripheral portions 546a and 546b may be configured to be folded over the central portion 545 of the electrode patch. Other locations of hinges on the substrate are within the scope of this disclosure. In some embodiments, the substrate may be folded over the bottom surface (i.e., the skin facing surface) such that the electrodes of the substrate are facing each other upon folding for, for example, protection of the electrodes. However, the disclosure is not so limiting, and the substrate may be folded over the top surface. In some implementations, the support such as the finger loops or finger cups may be used for holding the substrate during opening of a folded substrate and/or closing of a substrate from an open configuration.

Folding of the substrate allows for a multiple use or reusable electrode path. In some implementations, the substrate may be made of a durable and/or stiff material that allows for reusability such as, without limitation, plastic, steel, or other durable stiff material.

FIG. 6 illustrates an example electrode patch 640 that is similar to the electrode patch 140 but including structural elements comprising finger cups 647a and b, and a flap 649. To facilitate description, in the following embodiments of the disclosure, the same or similar reference numerals are used to indicate the same or similar elements, and the description of the same technical contents is omitted. For the description of the omitted parts, reference may be made to the foregoing embodiments, which are not repeated herein.

Referring to FIG. 6, the finger cups 647a and b may be configured to act as a barrier to prevent over extension of fingers holding the electrode patch for placement such that the radius of curvature of the purlicue web space becomes larger than that required for correct placement of the electrode patch at the underside of a subject's breast. Specifically, finger cup 647a is configured to cup the thump of user and finger cup 647b is configured to cup the fingers of a user. Additionally, a flap 649 is provided that extends over the hand of a user holding the electrode patch. The flap may be permanently attached to the substrate 642 (and/or may be removable) at one or more locations along the inner edge 645. In some embodiments, the flap 649 may support the breast of a subject when extending over the hand of the subject during positioning of the electrode patch on a subject (and/or after placement of the electrode patch). The flap may, optionally, be configured (i.e., positioned and/or sized) for creating and/or maintaining correct positioning and shape of the purlicue web space (similar to 334) and/or provide a convertible lip for resting the breast of a subject (similar to 234).

Optionally, one or more sensors or electrodes may be included on the underside 649a (i.e., the side facing the hand of the subject) of the flap 649. For example, a temperature sensor and/or an electrode may be included on the underside of the flap.

In some embodiments, one or more sensors and/or electrodes may be provided on the top surface of the substrate 642. For example, a pulse oximeter sensor 691 may be included on the top surface substrate 642 at or proximal to the finger loop 647a for collecting oxygen saturation data from a thumb of the subject. The location of the pulse oximeter sensor 691 may be optimized to prevent variation due to skin color, nail paint etc. in the collected data. For example, the pulse oximeter sensor 691 may be positioned to be proximal to a thumb pad of the subject. In another example embodiment, an electrode 692 (e.g., the right limp electrode) may be included on the top surface of the substrate 642 at or proximal to the finger loop 647b. This allows for non-concurrent measurement of ECG signals from various limbs of the user as the user can move the electrode patch to different locations (e.g., top of right leg, top of left leg, etc.) for collecting ECG signals before or after collection of pericardium ECG signals. In certain other embodiments, a temperature sensor 693 may be included on the top surface of the substrate 642 in a location that allows for accurate collection of temperature data (e.g., from the palm of a user).

Optionally, a box 694 may be provided on the substrate 642 to include one or more sensors and/or electrodes. For example, an accelerometer may be included for measuring the breathing rate of a subject.

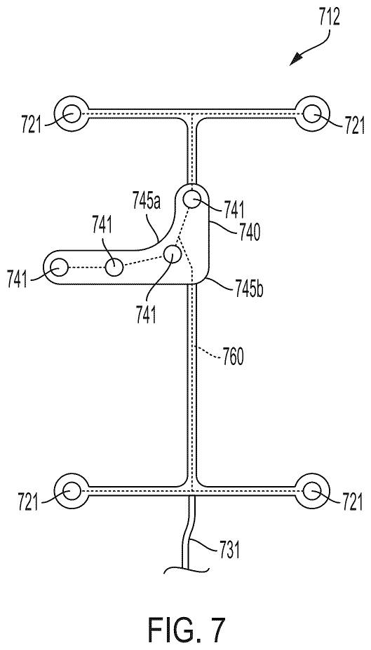

Referring to FIG. 7, an alternative embodiment of an ECG lead system 712 is shown. The ECG lead system is similar to the ECG lead system 12 but includes four limb electrodes 721 rather than including a limb electrode on a band, for example, the data collection device. The ECG lead system 712 includes electrodes 741 mounted on a substrate of an electrode patch 740. The limb electrodes and electrodes on the electrode patch are connected via electrical pathways 760. The electrode patch 740 is similar to the electrode patch 140 except the electrode patch 740 includes a corner outer surface 745b rather than a curved portion. The corner outer surface 745b has a smaller radius than the concave edge 745a. Additionally, in this example, a wire 731 extends out of the portion between limb electrodes 721. In some embodiments, the ECG lead system 712 could be used for connecting to a data collection device that is not arm mounted and does not include a band electrode. Additionally, the substrate is not attached to a pulse oximeter and only carries electrodes. In some embodiments, a pulse oximeter or any other sensor could be attached to a data collection device separately.

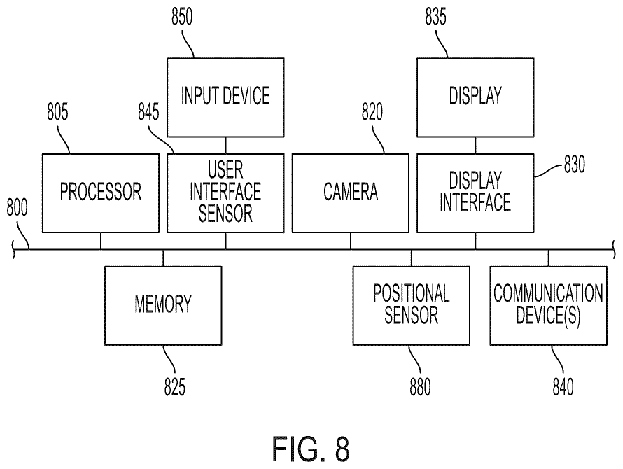

FIG. 8 depicts an example of internal hardware that may be included in any of the electronic components of the system, such as the electrode patch, the data collection device, the sensors, the user device, or a local or remote computing device in the system. An electrical bus 800 serves as a communication path via which messages, instructions, data, or other information may be shared among the other illustrated components of the hardware. Processor 805 is a central processing device of the system, configured to perform calculations and logic operations required to execute programming instructions. As used in this document and in the claims, the terms “processor” and “processing device” may refer to a single processor or any number of processors in a set of processors that collectively perform a set of operations, such as a central processing unit (CPU), a graphics processing unit (GPU), a remote server, or a combination of these. Read only memory (ROM), random access memory (RAM), flash memory, hard drives and other devices capable of storing electronic data constitute examples of memory devices 625. A memory device may include a single device or a collection of devices across which data and/or instructions are stored.

An optional display interface 830 may permit information to be displayed on a display device 835 in visual, graphic, or alphanumeric format. An audio interface and audio output (such as a speaker) also may be provided. Communication with external devices may occur using various communication devices 840 such as a wireless antenna, a radio frequency identification (RFID) tag and/or short-range or near-field communication transceiver, each of which may optionally communicatively connect with other components of the device via one or more communication systems. The communication device 840 may be configured to be communicatively connected to a communications network, such as the Internet, a local area network or a cellular telephone data network.

The hardware may also include a user interface sensor 845 that allows for receipt of data from input devices 850 such as a keyboard, a mouse, a joystick, a touchscreen, a touch pad, a remote control, a pointing device and/or microphone. Digital image frames also may be received from a camera 820 that can capture video and/or still images. The system also may include a positional sensor 880 to detect position and movement of the device. Examples of positional sensors 880 include a global positioning system (GPS) sensor device that receives positional data from an external GPS network.

Based on the teachings contained in this disclosure, it will be apparent to persons skilled in the relevant art(s) how to make and use embodiments of this disclosure using data processing devices, computer systems and/or computer architectures other than that shown in FIG. 8. In particular, various embodiments can operate with software, hardware, and/or operating system implementations other than those described in this document.

### Kits

The disclosure also includes a kit comprising components useful within the methods of the disclosure and instructional material that describes, for instance, the method of using the electrode patch as described elsewhere herein. The kit may comprise components and materials useful for performing the methods of the disclosure. For instance, the kit may comprise one or more of an electrode patch, a data collection device, a user device, one or more sensors, electric connectors, arm band, etc. of the current disclosure. In other embodiments, the kit may further comprise software and electronic equipment. The software and electronic equipment may be presented in a compact form for portable use.

In certain embodiments, the kit comprises instructional material. Instructional material may include a publication, a recording, a diagram, or any other medium of expression which can be used to communicate the usefulness of the device described herein. The instructional material of the kit of the disclosure may, for example, be affixed to a package which contains one or more instruments which may be necessary for the desired procedure. Alternatively, the instructional material may be shipped separately from the package, or may be accessible electronically via a communications network, such as the Internet.

In one embodiment, the disclosure includes a kit for portable use.

Terminology that is relevant to this disclosure includes:

An “electronic device” or a “computing device” refers to a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, gaming systems, televisions, digital home assistants and mobile electronic devices such as smartphones, fitness tracking devices, wearable virtual reality devices, Internet-connected wearables such as smart watches and smart eyewear, personal digital assistants, cameras, tablet computers, laptop computers, media players and the like. Electronic devices also may include appliances and other devices that can communicate in an Internet-of-things arrangement, such as smart thermostats, refrigerators, connected light bulbs and other devices. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion above, a client device, server device, virtual machine or container may be referred to simply as a “device” for brevity. Additional elements that may be included in electronic devices that are data collection devices are discussed above in the context of FIG. 8.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices. A memory may contain programming instructions that are configured to cause a processor to execute any of the actions described above in this document. A computer program product is a memory device with programming instructions stored on it.

In this document, the term “transceiver” refers to a device that includes an antenna and other components that can transmit data to and/or receive data from one or more other devices via a wireless communication path.

In this document, the terms “communication link” means a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices. The network may include or is configured to include any now or hereafter known communication networks such as, without limitation, a BLUETOOTH® communication network, a Z-Wave® communication network, a wireless fidelity (Wi-Fi) communication network, a ZigBee communication network, a HomePlug communication network, a Power-line Communication (PLC) communication network, a message queue telemetry transport (MQTT) communication network, a MTConnect communication network, a cellular network a constrained application protocol (CoAP) communication network, a representative state transfer application protocol interface (REST API) communication network, an extensible messaging and presence protocol (XMPP) communication network, a cellular communications network, any similar communication networks, or any combination thereof for sending and receiving data. As such, the network may be configured to implement wireless or wired communication through cellular networks, WiFi, BlueTooth, Zigbee, RFID, BlueTooth low energy, NFC, IEEE 802.11, IEEE 802.15, IEEE 802.16, Z-Wave, Home Plug, global system for mobile (GSM), general packet radio service (GPRS), enhanced data rates for GSM evolution (EDGE), code division multiple access (CDMA), universal mobile telecommunications system (UMTS), long-term evolution (LTE), LTE-advanced (LTE-A), MQTT, MTConnect, CoAP, REST API, XMPP, or another suitable wired and/or wireless communication method. The network may include one or more switches and/or routers, including wireless routers that connect the wireless communication channels with other wired networks (e.g., the Internet). The data communicated in the network may include data communicated via short messaging service (SMS), multimedia messaging service (MMS), hypertext transfer protocol (HTTP), direct data connection, wireless application protocol (WAP), e-mail, smart energy profile (SEP), ECHONET Lite, OpenADR, MTConnect protocol, or any other protocol.

As used herein, the term “user device” refers to any general-purpose computing device capable of data processing. In one example, the user device can be a mobile device, such as a smartphone (e.g., iPhone®, Android®-enabled phone, etc.), a personal digital assistant (PDA), a tablet, an e-reader, fitness tracking devices, smartwatches, wearable virtual reality devices, Internet-connected wearables such as smart watches and smart eyewear, or other mobile or portable computing devices. In another example, the user device can be a personal computing device, such as a desktop, a laptop, or other wired and wireless personal computers.

It will be understood that various modifications may be made to the embodiments disclosed herein. Likewise, the above disclosed methods may be performed according to an alternate sequence. Therefore, the above description should not be construed as limiting, but merely as exemplification of the various embodiments. Those skilled in the art will envision other modifications within the scope and spirit of the claims appended hereto.

What is claimed is:

1. An electrode patch, comprising:
a substrate that includes a base surface; and
a plurality of electrodes mounted with respect to the base surface and configured to collect electrocardiogram (ECG) measurements from a subject when the base surface is placed against a precordium of the subject;
wherein the substrate includes a central portion including a concave side that is shaped and dimensioned when the base surface is placed against a precordium of the subject.

2. The electrode patch of claim 1, wherein the concave side has a radius of curvature that is similar to a radius of curvature of webspace between a thumb and an index finger of a hand of the subject when the hand is open.

3. The electrode patch of claim 1, wherein the concave side has a radius of curvature that is between about 1 to 4 inches.

4. The electrode patch of claim 1, further comprising a first peripheral portion and a second peripheral portion that flank the central portion.

5. The electrode patch of claim 4, wherein the first peripheral portion and the second peripheral portion flank the central portion to form an approximate L-shape.

6. The electrode patch of claim 4, wherein the first peripheral portion has a length that is similar to an average size of a thumb of a human subject, and the second peripheral portion has a length similar to an average size of a finger of the human subject.

7. The electrode patch of claim 1, wherein the concave edge has an arc angle configured to at least partially concentrically align with an underside of a breast of the subject.

8. The electrode patch of claim 1, wherein the electrode patch includes exactly four electrodes for collection of ECG measurements from the following precordial locations: V2, V3, V4, and V5.

9. The electrode patch of claim 1, wherein the electrode patch is expandable to include at least one more electrode for collection of ECG measurements from one or more of the following precordial locations: V1 and V6.

10. The electrode patch of claim 1, further comprising a support configured to allow the subject to hold the electrode patch in place against at least a portion of the precordium of the subject.

11. The electrode patch of claim 1, further comprising a structural element configured to allow the subject to correctly position webspace between a thumb and an index finger of a hand of the subject when the hand is open while holding the electrode patch in place.

12. The electrode patch of claim 1, further comprising a comfort lip extending from the concave side and that provides padding to the breast of the subject upon placement of the concave side under the breast of the subject.

13. The electrode patch of claim 12, wherein the comfort lip is further configured to extend at least partially over a webspace between a thumb and an index finger of a hand of the subject for holding or positioning the electrode patch under the breast of the subject.

14. The electrode patch of claim 1, wherein the substrate comprises one or more hinges for folding at least two portions of the substrate on top of each other.

15. An electrode patch comprising:
a substrate configured for placement against at least a portion of a precordium of a subject;
a support connected to the substrate and configured to naturally assume a curvature in an unloaded state; and
a plurality of electrodes mounted on the substrate and configured for collecting electrocardiogram (ECG) measurements from the subject.

16. The electrode patch of claim 15, wherein the support includes a loop configured and dimensioned to receive one or more fingers of the hand of the subject.

17. The electrode patch of claim 15, wherein the support is a comfort lip extending from a surface of the substrate and is configured to cover at least the portion of a hand of the subject to provide padding to a breast of the subject.

18. The electrode patch of claim 17, wherein the comfort lip has an approximate U-shape, wherein a concave portion of the approximate U-shape is the hand contacting surface, and a convex portion of the approximate U-shape is the breast contacting surface.

19. An electrocardiogram (ECG) lead system, comprising:
up to four precordial electrodes, a number of the precordial electrodes being sufficient to generate a complete and accurate ECG of a subject;
one or more limb electrodes electrically coupled to the precordial electrodes; and
a plurality of electrical pathways connecting the precordial electrodes and the one or more limb electrodes;
wherein the precordial electrodes and the one or more limb electrodes are configured to electrically connect to a data collection device which collects data from each of the electrodes.

20. The ECG lead system of claim 19, wherein the ECG lead system comprises the following four precordial electrodes are: V2, V3, V4, and V5 electrodes.

21. A diagnostic system, comprising:
the ECG lead system of claim 19;
the data collection device electronically coupled to the ECG lead system;
a user device in electronic communication with the data collection device; and
a network in electronic communication with the user device and a remote server.

22. An electrode patch, comprising:
an L-shaped substrate; and
a plurality of electrodes mounted on a base surface of the substrate and configured to collect electrocardiogram (ECG) measurements from a subject,
wherein the substrate comprises at least one structural element oriented facing away from the base surface.

23. The electrode patch of claim 22, wherein the at least one structural element is configured to allow the subject to hold the electrode patch against a pericardium of the subject.

24. The electrode patch of claim 22, wherein the at least one structural element includes one or more flaps extending from an inside edge of the L-shaped substrate, the one or more flaps configured to extend at least partially over a webspace between a thumb and an index finger of a hand of the subject for holding or positioning the electrode patch under the breast of the subject.

25. The electrode patch of claim 22, wherein the at least one structural element is a comfort lip configured to provide padding to a breast of the subject upon placement of the L-shaped substrate around a breast of the subject.

26. The electrode patch of claim 22, wherein the at least one structural element includes a finger cup configured and dimensioned to receive a thumb of the subject, the finger cup included in a first arm of the L-shaped substrate.

27. An electrode patch, comprising:
a substrate that includes a base surface;
a plurality of electrodes mounted with respect to the base surface and configured to collect electrocardiogram (ECG) measurements from a subject when the base surface is placed against a precordium of the subject; and
at least one electrode mounted on a hand facing surface of the substrate, the hand facing surface being in contact with a hand of the subject when holding the base surface against the pericardium of the subject.

28. The electrode patch of claim 27, further comprising at least one sensor mounted on the hand facing surface.

29. A diagnostic method for collecting electrocardiogram (ECG) data from a subject, the method comprising:
providing an ECG lead system including up to four precordial electrodes mounted on a substrate, a number of the precordial electrodes being sufficient to generate a complete and accurate ECG of a subject;
positioning a concave side the substrate around an underside of a breast of the subject such that the pericardial electrodes are correctly positioned for collecting the ECG data;
receiving, at a data collection device ECG measurement data from one or more of the precordial electrodes; and
transmitting, from the data collection device, to a remote server, the ECG measurement data.
